(19) United States
(12) Patent Application Publication
(10) Pub. No.: US 2017/0344659 A1
(43) Pub. Date: Nov. 30, 2017

(54) METHOD FOR CLASSIFYING DATA, DATA CLASSIFICATION APPARATUS, AND MEDIUM

(71) Applicants: FUJITSU LIMITED, Kawasaki-shi (JP); RIKEN, Wako-shi (JP)

(72) Inventors: Daisuke Kushibe, Kawasaki (JP); Tsuyoshi Esaki, Wako (JP); Tsutomu Masujima, Wako (JP); Masahito Yamaguchi, Moriya (JP)

(73) Assignees: FUJITSU LIMITED, Kawasaki-shi (JP); RIKEN, Wako-shi (JP)

(21) Appl. No.: 15/601,004

(22) Filed: May 22, 2017

(30) Foreign Application Priority Data
May 24, 2016 (JP) 2016-103425

(51) Int. Cl. G06F 17/30; H01J 49/00; G01N 33/483

(52) U.S. Cl. CPC G06F 17/30946; G01N 33/4833; H01J 49/0036

(57) ABSTRACT

A method for classifying data executed by a computer includes obtaining a plurality of data groups, each of the data groups including a plurality of data items about detected intensities being associated with physical index values, respectively; and classifying, based on identification information of each of the data groups and the physical index values, the data items included in the data groups into a plurality of clusters.

### CROSS-REFERENCE TO RELATED APPLICATIONS

This application is based upon and claims the benefit of priority of the prior Japanese Priority Application No. 2016-103425 filed on May 24, 2016, the entire contents of which are hereby incorporated by reference.

### FIELD

The present disclosure relates to a method for classifying data, a data classification apparatus, and a medium.

### BACKGROUND

Mass spectroscopes have been used for investigating substances (molecules) included in a sample. A mass spectroscope uses, for example, a property of a substance that when the substance ionized in a vacuum to which a high voltage is applied, flies in the mass spectroscope by electrostatic force, an electromagnetic effect is applied to the substance along the flight path, which causes the substance to be separated in a direction perpendicular to the flight direction depending on it s mass-to-charge ratio (m/z). Then, the mass spectroscope detects the amount of the arrived substance (ions) for each substance, to obtain multiple data items where each item is a pair of a mass-to-charge ratio and a detected intensity (which may be simply referred to as the “intensity”, below). Data contents obtained as such or a graph of the data where the horizontal axis represents the mass-to-charge ratio and the vertical axis represents the detected intensity is called an “MS spectrum (mass spectrum)”. Note that the resolution of the mass-to-charge ratio in raw data output from the mass spectroscope is higher than a resolution with which the difference of the mass-to-charge ratios of substances to be measured can be distinguished. Therefore, there may be cases where peaks are detected on a waveform obtained by connecting the detected intensities in the raw data (peak picking), to convert the data items into pairs of mass-to-charge ratios and detected intensities for the detected peaks. The data after having such peak picking applied to is also called an MS spectrum, or a peak-picked MS spectrum.

Note that although a set of raw data items of an MS spectrum may be obtained by one-time measurement by the mass spectroscope, it is difficult to assure the precision by the one-time measurement. Therefore, it is common to measure the same sample multiple times under the same condition by the mass spectroscope. The raw data corresponding to MS spectrums obtained by multiple-time measurement where the count of measurement is the same as the number of the MS spectrums, is used for identifying multiple peaks each of which has the same mass-to-charge ratio, so as to average the detected intensities of the identified multiple peaks. Patent documents 1-3 have been know as information processing technologies in mass spectrometry.

### RELATED-ART DOCUMENTS

### Patent Documents

[Patent Document 1] Japanese Unexamined patent Application Publication No. 2014-112068

[Patent Document 2] Japanese Unexamined Patent Application Publication No. 2013-40808

[Patent Document 3] Japanese Unexamined Patent Application Publication No. 2012-247198

### SUMMARY

According to an embodiment, a method for classifying data executed by a computer includes obtaining a plurality of data groups, each of the data groups including a plurality of data items about detected intensities being associated with physical index values, respectively; and classifying, based on identification information of each of the data groups and the physical index values, the data items included in the data groups into a plurality of clusters.

The object and advantages of the embodiment will be realized and attained by means of the elements and combinations particularly pointed out in the claims. It is to be understood that both the foregoing general description and the following detailed description are exemplary and explanatory and are not restrictive of the invention as claimed.

### BRIEF DESCRIPTION OF DRAWINGS

FIG. 1 is a diagram illustrating a configuration examples of a system according to an embodiment;

FIG. 2 is a diagram illustrating an example of a hardware configuration of an information processing apparatus;

FIG. 3 is a diagram illustrating an example of a hardware configuration of an information processing apparatus;

FIG. 4 is a flowchart illustrating an example of a process according to an embodiment;

FIG. 5 is a diagram illustrating an example of a data structure of raw data;

FIGS. 6A-6B are diagrams illustrating an example of peak picking;

FIG. 7 is a diagram illustrating an example of a data structure of peak-picked MS spectrums;

FIG. 8 is a diagram illustrating an example of alignment;

FIG. 9 is a flowchart illustrating an example of a process of alignment;

FIG. 10 is a flowchart illustrating an example of a process of cluster decomposition;

FIG. 11 is a flowchart illustrating an example of a process of peak cluster decomposition;

FIG. 12 is a diagram illustrating an example of data series;

FIG. 13 is a diagram illustrating an example of cluster decomposition;

FIG. 14 is a diagram illustrating an example of peak cluster decomposition;

FIG. 15 is a diagram illustrating an example of a result of alignment; and

FIGS. 16A-16C are diagrams illustrating an example of noise removal.

### DESCRIPTION OF EMBODIMENTS

If precise analysis is required, even if multiple peaks are identified, each of which has the same mass-to-charge ratio, it may not be possible to identify a peak corresponding to the same substance correctly due to an influence of fluctuation of the measured values. In other words, if such an influence of the fluctuation is not taken into consideration, the precision of the mass spectrometry may drop. Therefore, in order to raise the precision of the mass spectrometry, it is important to classify correctly the data of multiple peaks obtained by multiple-time measurement for each of the substances. Here, although the MS spectrum is taken as an example for the description, the same problem arises, not only when processing the MS spectrums, but also when processing discrete spectrums, for example, optical spectrums (including infrared spectrums, ultraviolet spectrums, etc.) and nuclear magnetic resonance spectrums.

In the following, preferable embodiments will be described.

According to an embodiment, it is possible to raise the precision of data classification.

<Configuration>

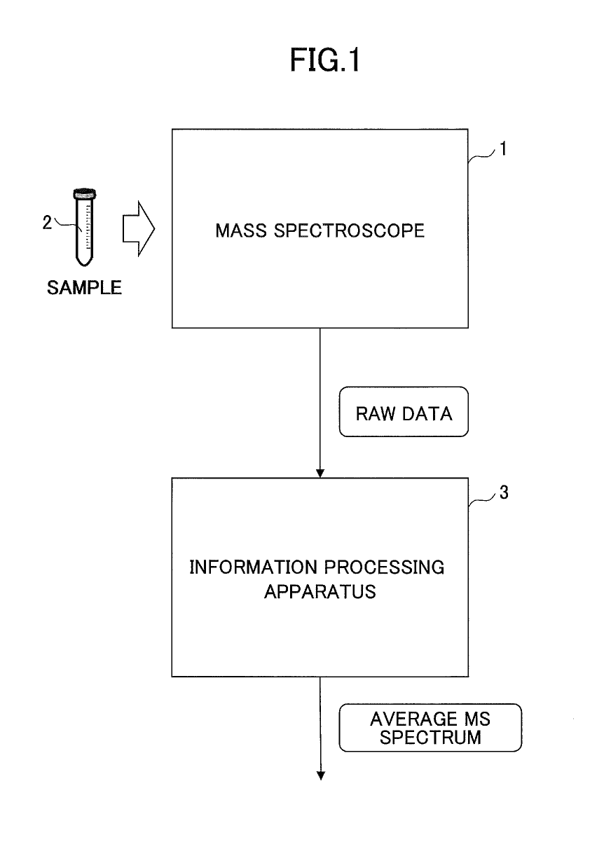

FIG. 1 is a diagram illustrating a configuration example of a system according to an embodiment. In FIG. 1, the system includes a mass spectroscope 1 and an information processing apparatus 3. The mass spectroscope 1 measures (applies mass spectrometry to) a sample 2, and outputs raw data of an MS spectrum (data group) including multiple data items of pairs of mass-to-charge ratios (m/z) and detected intensities (may be simply referred to as the “intensity”). The raw data may include, in addition to the MS spectrum, other information such as measurement conditions. Note that in general, measurement is executed multiple times for the same sample 2, and the MS spectrum for each time of the measurement can be distinguished from the other spectrums in the raw data. The information processing apparatus 3 processes information by reading offline or online the raw data output by the mass spectroscope 1, and eventually outputs an average MS spectrum in a data format or a graph format. Note that the information processing apparatus 3 is not limited to be constituted with a single unit, but may be constituted with multiple units.

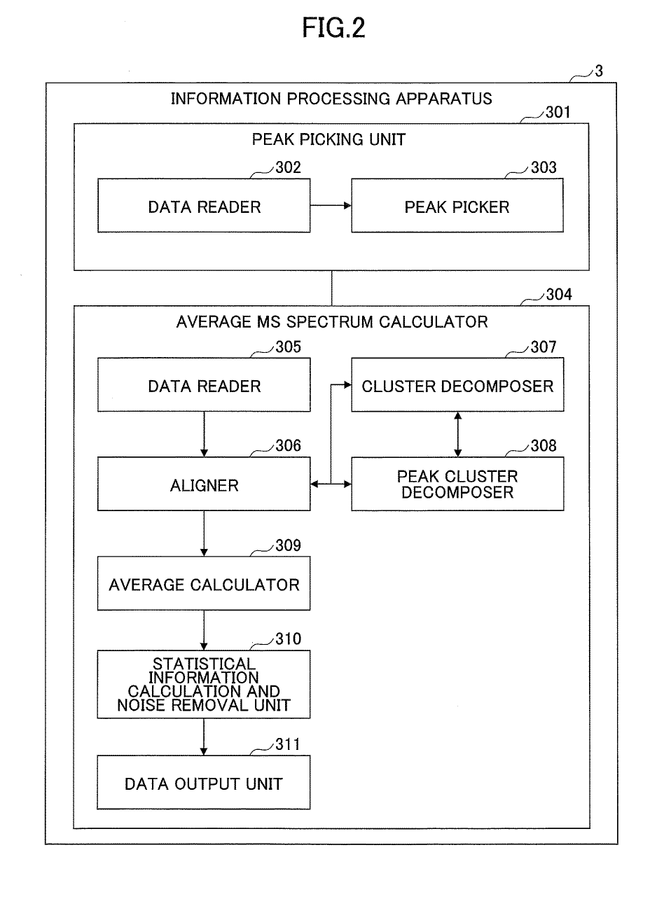

FIG. 2 is a diagram illustrating a software configuration example of the information processing apparatus 3. In FIG. 2, the information processing apparatus 3 includes a peak picking unit 301 and an average MS spectrum calculator 304. The peak picking unit 301 has a function to execute a peak-picking process, and includes a data reader 302 and a peak picker 303. The data reader 302 has a function to read the raw data output by the mass spectroscope 1. The peak picker 303 has a function to execute peak picking on the raw data read by the data reader 302, and to output the peak-picked MS spectrum data. Note that peak picking is applied to an MS spectrum of measurement of one time distinguished in the raw data, and a spectrum number is given to the peak-picked MS spectrum data as identification information of the MS spectrum, to be included in the output data.

The average MS spectrum calculator 304 has a function to calculate an average MS spectrum. The average MS spectrum calculator 304 includes a data reader 305, an aligner 306, a cluster decomposer 307, a peak cluster decomposer 308, an average calculator 309, a statistical information calculation and noise removal unit 310, and a data output unit 311. The data reader 305 has a function to read the peak-picked MS spectrum data, which has been output by the peak picking unit 301. The aligner 306 has a function to identify (align) data items of corresponding peaks in multiple MS spectrums, taking fluctuation of the measured values into account, in the multiple peak-picked MS spectrum data, and having been read by the data reader 305.

The cluster decomposer 307 has a function to execute cluster decomposition when called from the aligner 306 or the peak cluster decomposer 308. The cluster decomposition is a process that is applied to a data group (data series) to be processed, in which all the data included in multiple MS spectrums has been sorted by the mass-to-charge ratio, so that if the difference of the mass-to-charge ratios between adjacent data items is less than or equal to a predetermined permissible value, the data items are put into the same point set, or if the difference is greater than the permissible value, the data items are put into different point sets. This process classifies (applies clustering to) the data series depending on whether the difference of the mass-to-charge ratios is within the predetermined permissible value. Therefore, the data items having the same spectrum number may be included in a single point set. However, the data items of the same spectrum number being included in a single point set, imply that the data items that should be essentially recognized as different peaks are classified into the same point set. Therefore, a point set not including data items having the same spectrum number will be referred to as a “peak cluster”, and a point set including data items having the same spectrum number will be referred to as a “semi-cluster”, to be distinguished. The peak cluster decomposer 308 has a function to execute peak cluster decomposition for a semi-cluster when called from the aligner 306. The peak cluster decomposition is a process to decompose a semi-cluster into peak clusters.

The average calculator 309 has a function to calculate the average of mass-to-charge ratios and the average of detected intensities, of the data items included in each peak cluster, based on a process result of the aligner 306. Since data items put into each peak cluster are identified as corresponding peaks in multiple MS spectrums, the average calculator 309 is provided to obtain the averages representing the data items. The statistical information calculation and noise removal unit 310 calculates a detection frequency (observation probability) from the ratio of the number of the data items included in each cluster with respect to the number of MS spectrums, as one of the statistical information items. It is possible to use the detection frequency as information about evaluation of the data. For example, if the same number of data items as the number of MS spectrums are included in a cluster, the detection frequency is 100%, which means the corresponding peak is detected in very MS spectrum. Therefore, the data can be considered highly reliable. On the contrary, if a lower number of data items are included in a cluster than the number of MS spectrums, the detection frequency takes a smaller value. This may be caused by a very small amount of impurities creeping into the mass spectroscope 1 in a non-reproducible way, electric noise, and the like, and hence, the data may be considered to have a low reliability.

The statistical information calculation and noise removal unit 310 also has a function to remove a peak cluster including unreliable data items as noise or the like, based on the detection frequency. The data output unit 311 has a function to output a data group that includes data items of pairs of the average of the mass-to-charge ratios and the average of the detected intensities of each peak cluster after noise has been removed, as data of the average MS spectrum. Note that the data output unit 311 may have a function to output the average MS spectrum processed into a graph format.

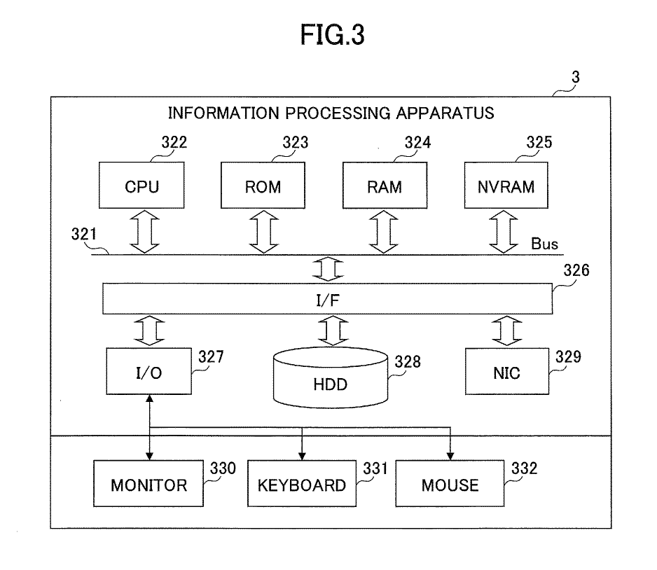

FIG. 3 is a diagram illustrating an example of the hardware configuration of the information processing apparatus 3. In FIG. 3, the information processing apparatus 3 includes a CPU (Central Processing Unit) 322 connected to a system bus 321, a ROM (Read-Only Memory) 323, a RAM (Random Access Memory) 324, and a NVRAM (Non-Volatile Random Access Memory) 325. The information processing apparatus 3 also includes an I/F (Interface) 326 connected with an I/O (Input/Output Device) 327, and HDD (Hard Disk Drive) 328, a NIC (Network Interface Card) 329; and a monitor 330, a keyboard 331, and a mouse 332 connected to the I/O 327. A CD/DVD (Compact Disk/Digital Versatile Disk) drive or the like may be connected to the I/O 327.

The units illustrated in FIG. 2 are implemented by a program running on the CPU 322 in FIG. 3. The program may be provided on a recording medium, may be provided via a network, or may be installed in the ROM.

<Operation>

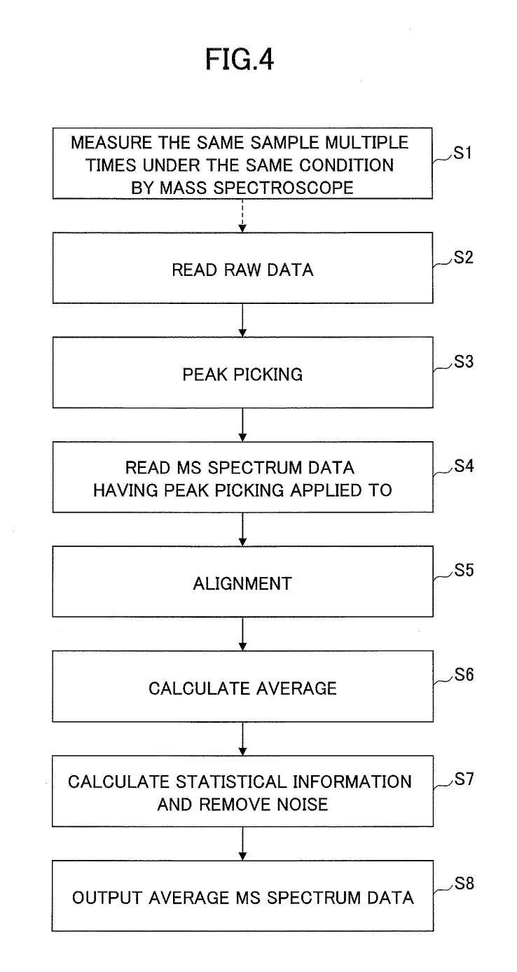

FIG. 4 is a flowchart illustrating an example of a process in the embodiment. In FIG. 4, a person in charge of measurement performs measurement of the same sample 2 multiple times under the same measurement conditions by using the mass spectroscope 1 (Step S1). As a measurement result, raw data is output.

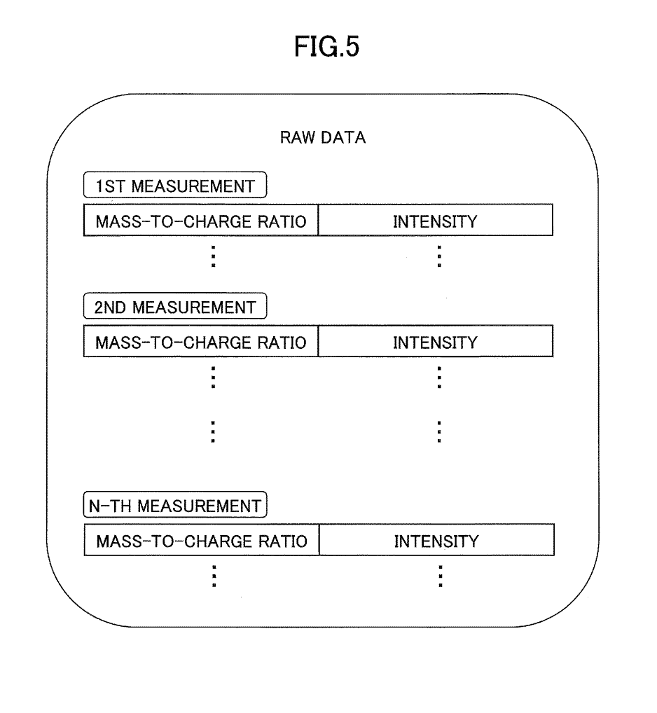

Next, the data reader 302 of the information processing apparatus 3 reads offline or online the raw data output by the mass spectroscope 1 (Step S2). FIG. 5 is a diagram illustrating an example of the data structure of the raw data that includes multiple data items of pairs of mass-to-charge ratios and detected intensities for the first measurement to the N-th measurement.

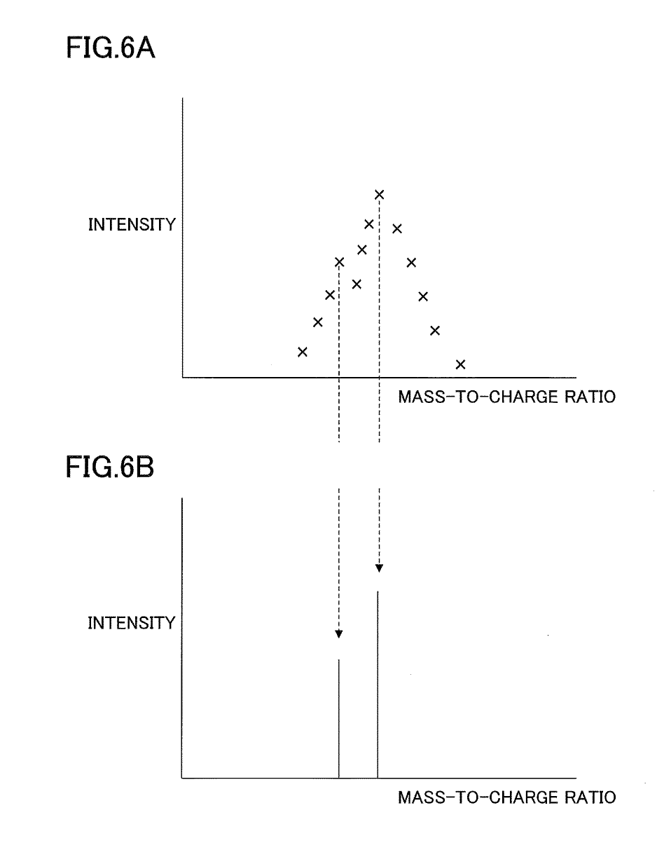

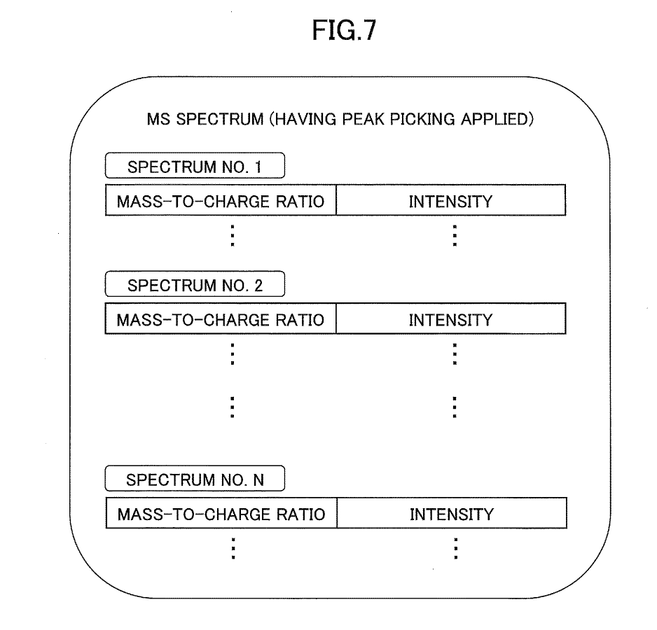

Referring back to FIG. 4, the peak picker 303 of the information processing apparatus 3 executes peak picking on the raw data read by the data reader 302, and outputs the peak-picked MS spectrum data (Step S3). FIGS. 6A-6B are diagrams illustrating an example of peak picking; FIG. 6A illustrates raw data and FIG. 6B illustrates peaks identified by the peak picking. As illustrated in the figures, the peak picker 303 detects peaks in a waveform formed by connecting the detected intensities to one another in the raw data. Note that a known algorithm may be used for peak picking. It may be assumed that existing spectrum analysis software (ProteoWizard, etc.) is used in implementation. FIG. 7 is a diagram illustrating an example of a data structure of peak-picked MS spectrums that includes multiple data items of pairs of mass-to-charge ratios and detected intensities for the spectrum number 1 (corresponding to the first measurement) to the spectrum number N (corresponding to the N-th measurement).

Referring back to FIG. 4, the data reader 305 of the information processing apparatus 3 reads the peak-picked MS spectrum data that has been output by the peak picking unit 301 (Step S4).

next, on multiple items of the peak-picked MS spectrum data that has been read by the data reader 305, the aligner 306 identifies (aligns) corresponding peak data items in the multiple MS spectrums, taking fluctuations of the measured values into account (Step S5).

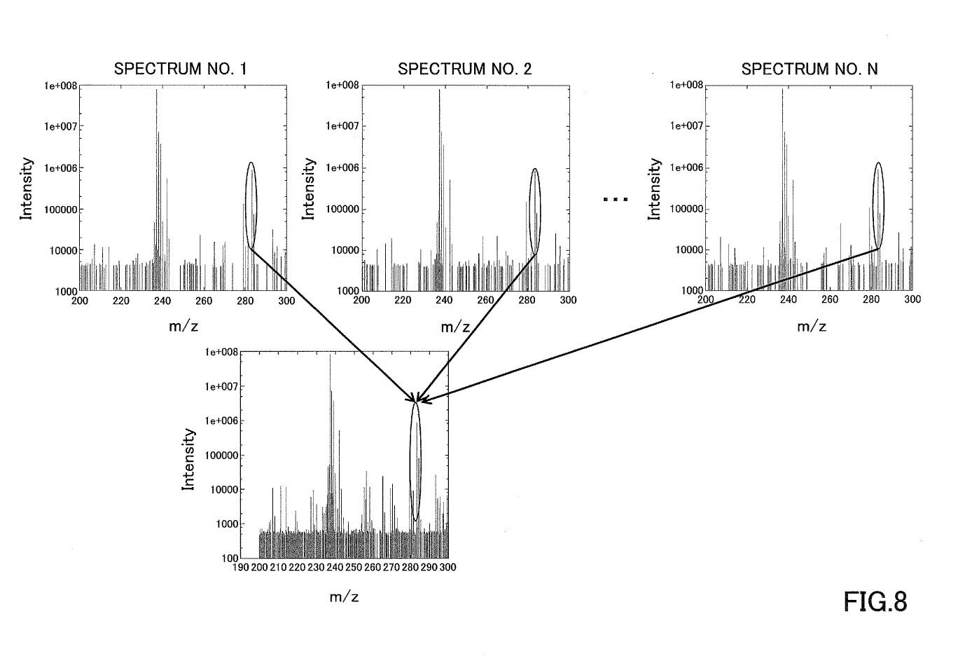

FIG. 8 is a diagram illustrating an example of alignment. In FIG. 8, due to fluctuation of the measured values, peak values of mass-to-charge ratios in the spectrum numbers 1, 2, . . . , N or the MS spectrums that may correspond to a certain single peak do not completely correspond to each other on the horizontal axis. In the present disclosure, a process is referred to as “alignment” that causes such peak values, which are considered to correspond to the same peak, to be associated with each other. The alignment is implemented by clustering technologies in the embodiment.

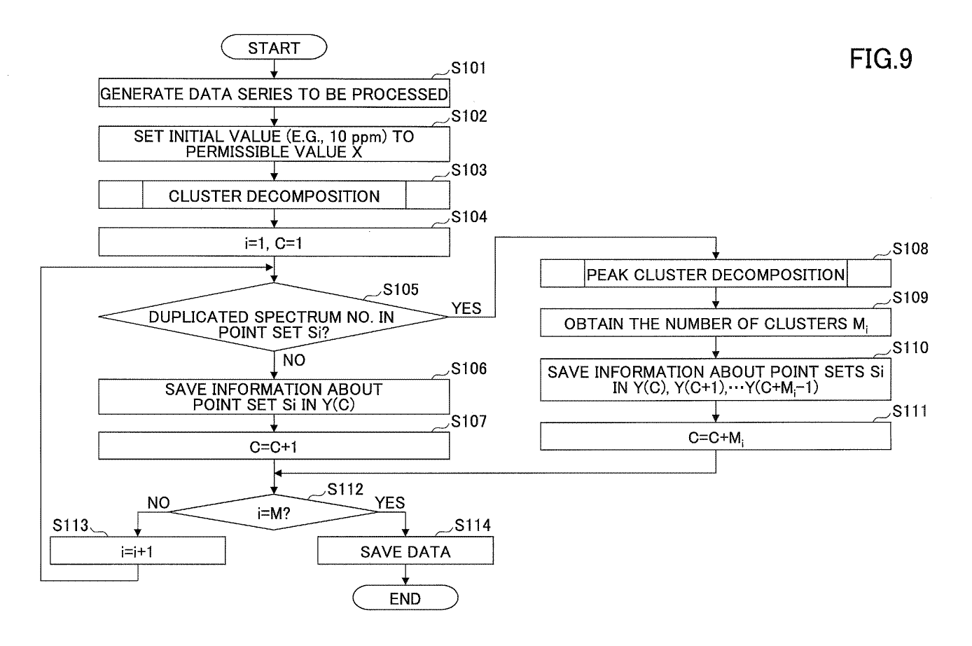

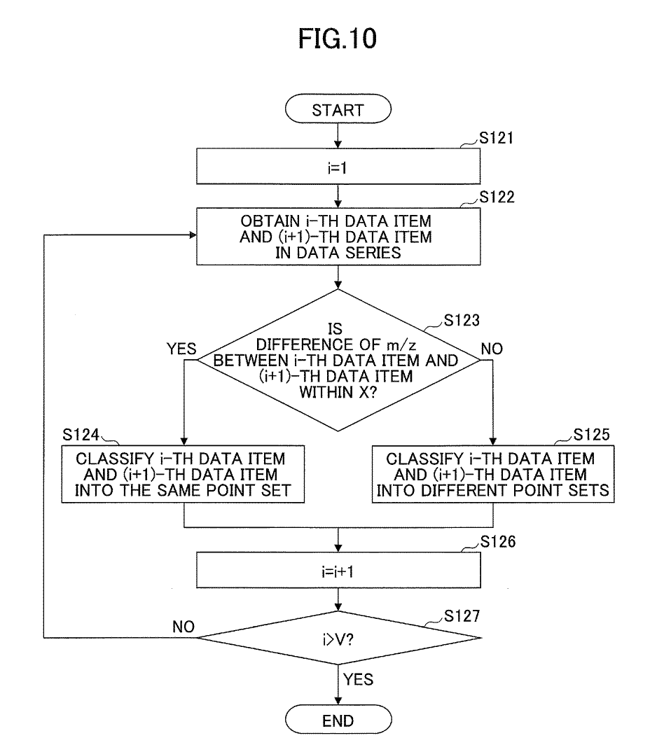

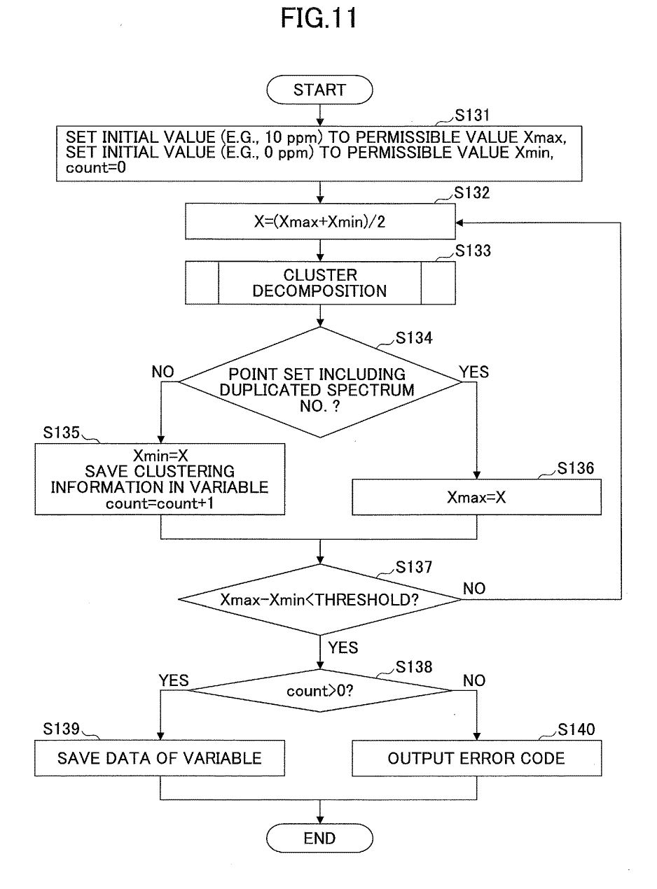

FIG. 9 is a flowchart illustrating an example of a process of alignment by the aligner 306. Further, FIG. 10 is a flowchart illustrating an examples of a process of cluster decomposition by the cluster decomposer 307 called from the aligner 306 or the peak cluster decomposer 308. FIG. 11 is a flowchart illustrating an example of a process of peak cluster decomposition by the peak cluster decomposer 308 called from the aligner 306.

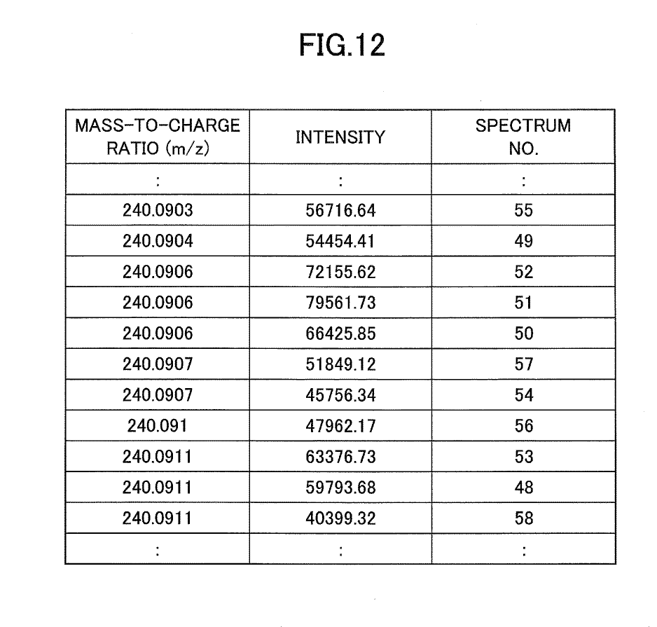

In FIG. 9, the aligner 306 creates data series to be processed from multiple items of peak-picked MS spectrum data that has been read by the data reader 306 (Step S101). In other words, the aligner 306 creates the data series by sorting all the data items included in the multiple peak-picked MS spectrums, by the mass-to-charge ratio. Assume that V represents the number of the data items included in the data series. FIG. 12 is a diagram illustrating an example of data series that include a list of data items each of which is a set of a mass-to-charge ratio (m/z), a detected intensity (intensity), and a spectrum number; and the data items are sorted by the mass-to-charge ratio (in this example, sorted in ascending order).

Referring back to FIG. 9, the aligner 306 sets a permissible value X, which is used for determining whether fluctuated mass-to-charge ratios correspond to the same peak, to an initial value (e.g., 10 ppm) (Step S102).

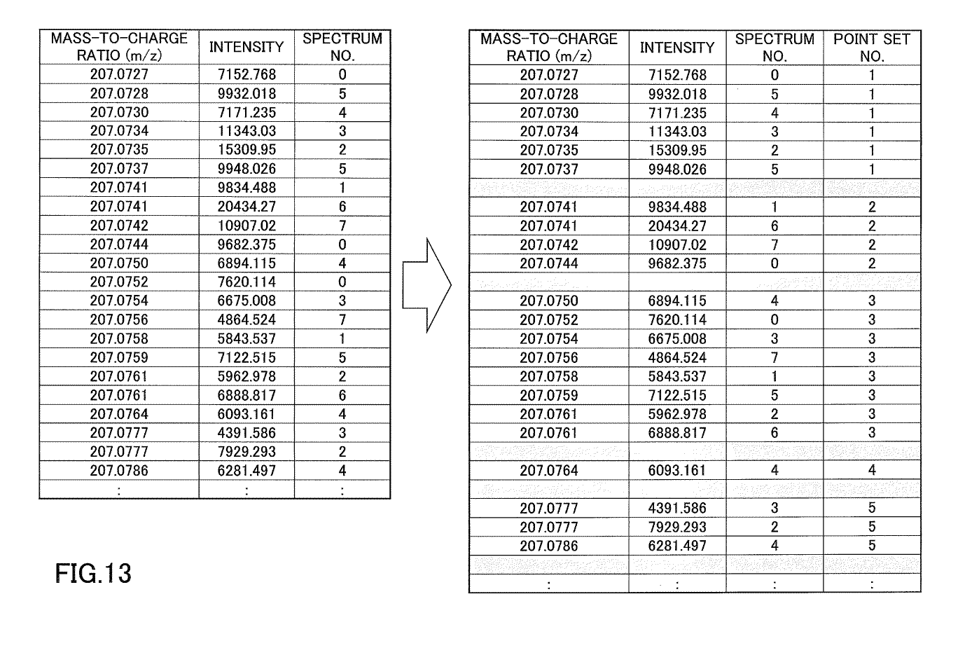

Next, the aligner 306 calls the cluster decomposer 307 and executes cluster decomposition (Step S103). The process of cluster decomposition will be described later in detail. The cluster decomposition causes the fluctuation range to be contained within the permissible value X, and classifies the data items into point sets that are separated from the adjacent sets by the permissible value X. Assume that M represents the number of the classified point sets. FIG. 13 is a diagram illustrating an example of cluster decomposition in which a table on the right is a result cluster decomposition applied to data series in a table on the left. A point set having the point set number “1” is a semi-cluster because data items are duplicated for the spectrum number “5”, and the other point sets are peak clusters.

Referring back to FIG. 9, and the aligner 306 sets the index i of a point set to an initial value “1”, and sets the peak cluster number C to an initial value “1” (Step S104).

Next, the aligner 306 determines whether a duplicated spectrum number exists in a point set Si identified by the index i (Step S105). If no duplication exists (NO at Step S105), the aligner 306 saves the information about the point set Si into a variable Y(C) to store the result of the peak cluster number C (Step S106). As the information about the point set Si, the aligner 306 may store mass-to-charge ratios and detected intensities of the data items included in the point set Si as they are, or may assign an identification number to the data and store the number if the mass-to-charge ratios and the detected intensities are to be stored in other are. Next, the aligner 306 increments the peak cluster number C (Step S107).

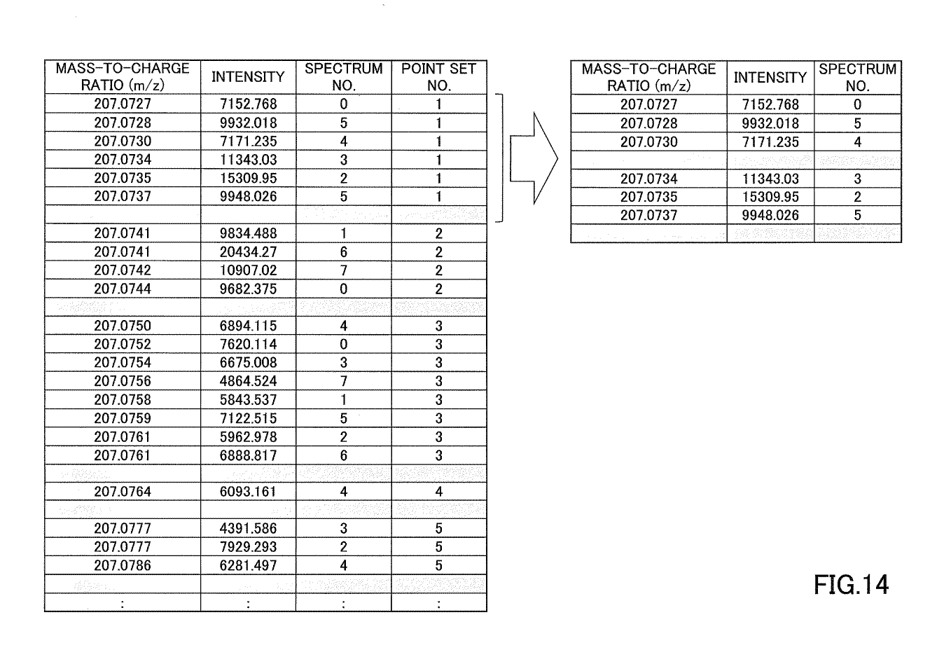

On the other hand, if a duplicated spectrum number exists in the point set Si (YES at Step S105), the aligner 306 calls the peak cluster decomposer 308 to apply peak cluster decomposition to the point set Si (Step S108). The process of peak cluster decomposition will be described later in detail. The peak cluster decomposition decomposes the point set Si being a semi-cluster into multiple peak clusters. FIG. 14 is a diagram illustrating an example of peak cluster decomposition in which the point set having the point set number “1” is decomposed into two peak clusters.

Referring back to FIG. 9, the aligner 306 obtains the number of the peak clusters Mᵢ obtained by the peak cluster decomposition (Step S109), and saves the information about the peak clusters in variables (Y(C), Y(C+1), . . . , and Y(C+Mᵢ−1) (Step S110). Next, the aligner 306 adds the number of the peak clusters Mᵢ to the peak cluster number C (Step S111).

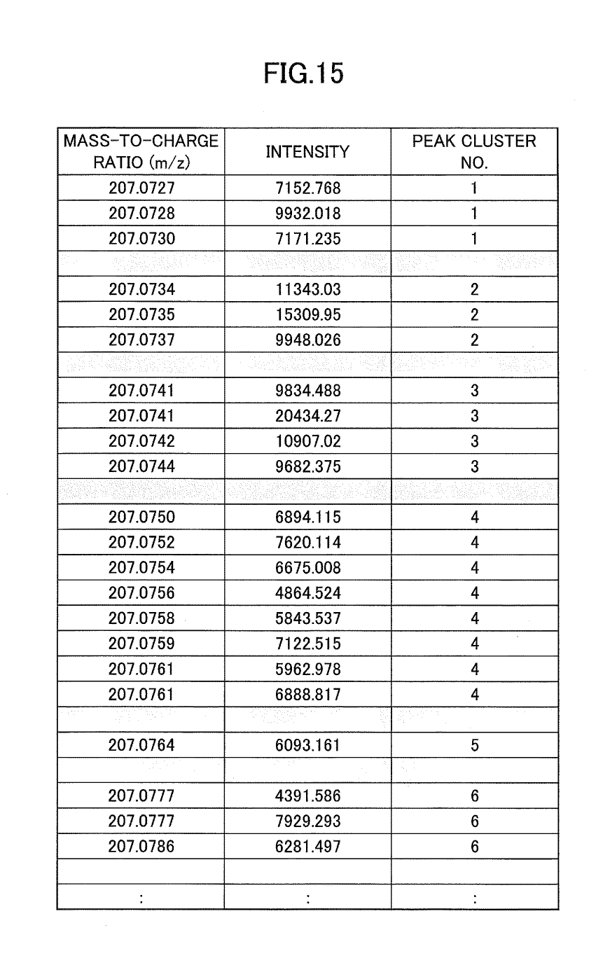

After having updated the peak cluster number C (Step S107 or S111), the aligner 306 determines whether the index i of the point set is equivalent to the number of the point sets M (Step, S112). If not equivalent (NO at Step S112), the aligner 306 increments the index i (Step S113), and returns to the determination of duplication in the point set Si (Step S105). If the index i of the point set is equivalent to the number of the point sets M (YES at Step S112), the aligner 306 saves the data of the variables Y into a storage area (Step S114), and ends the process. FIG. 15 is a diagram illustrating an example of a result of alignment saved in the storage area in which a peak cluster identified by the peak cluster number is associated with data items of mass-to-charge ratios and detected intensities.

Next, the process of cluster decomposition by the cluster decomposer 307 will be described in detail. In FIG. 10, the cluster decomposer 307 sets the index i to an initial value “1” (Step S121).

Next, the cluster decomposer 307 obtains the i-th data item and the (i+1)-th data item from the data series (Step S122), and determines whether the difference between m/z (mass-to-charge ratios) of the i-th data item and the (i+1)-th data item is less than the permissible value X (Step S123).

If less than the permissible value X (YES at Step S123), the cluster decomposer 307 classifies the i-th data item and the (i+1)-th data item into the same point set (Step S124). If not less than the permissible value X (NO at Step S123), the cluster decomposer 307 classifies the i-th data item and the (i+1)-th data item into different point sets (Step S125).

Next, the cluster decomposer 307 increments the index i (Step S126), and determines whether the index i exceeds the number of the data items of data series V (Step S127). If not exceeded (NO at Step S127), the cluster decomposer 307 returns to data obtainment (Step S122), or if exceeded (YES at Step S127), ends the process.

Next, the process of peak cluster decomposition by the peak cluster decomposer 308 will be described in detail. In FIG. 11, the peak cluster decomposer 308 sets the maximum permissible value Xmax to an initial value (e.g., 10 ppm), and sets the minimum permissible value Xmin to an initial value (e.g., 0 ppm), and sets the success count of peak cluster decomposition to an initial value “0” (Sept S131).

Next, the peak cluster decomposer 308 calculates the permissible value X by the following formula (Step S132).

X=(Xmax+Xmin)/2

Then, the peak cluster decomposer 308 calls the cluster decomposer 307 to execute cluster decomposition (Step S133). The process of cluster decomposition is as described in detail with reference to FIG. 10.

Next, the peak cluster decomposer 308 determines whether there is a point set that includes a duplicated spectrum number (Step S134). If no point set includes a duplicated spectrum number (NO at Step S134), the peak cluster decomposer 397 sets the minimum Xmin to the permissible value X at the current moment, saves the clustering information (information that represents which data item is classified into which point set) in the variables, and increments the success count (Step S135). If any point set includes a duplicated spectrum number (YES at Step S134), the peak cluster decomposer 308 sets the maximum Xmax to the permissible value X at the current moment (Step S136).

Next, the peak cluster decomposer 308 determines whether the difference between the maximum Xmax and the minimum Xmin is less than a predetermined threshold (e.g., 0.01 ppm) (Step S137). Then, if not less than the threshold (NO at Step S137), the peak cluster decomposer 308 determines that further optimization is possible, and returns to the calculation of the permissible value X (Step S132).

If the difference between the maximum Xmax and the minimum Xmin is less than the predetermined threshold (YES at Step S137), the peak cluster decomposer 308 determines whether the success count is greater than zero (greater than or equal to one) (Step S138). If the success count is greater than zero (YES at Step S138), the peak cluster decomposer 308 saves the data of the variables recording the clustering information in a storage area (Step S139), and ends the process. If the success cont is not greater than zero (equal to zero) (NO at Step S138), the peak cluster decomposer 308 outputs an error code representing that the peak cluster decomposition has failed (Step S140), and ends the process. Note that although the example has been described that uses a bisection method for optimization, another method (e.g., a Newton method) may be used for optimization.

In other words, although semi-clusters may be removed by making the permissible value X smaller to the utmost limit, if the permissible value X is too small, data items to be essentially classified as the same peak may be classified into different peak clusters. Therefore, the peak cluster decomposer 308 varies the permissible value X, to operate so as to obtain the maximum permissible value X in a range where a semi-cluster is not generated. Note that the distribution of peaks has a shape like a normal distribution, and the peaks have different dispersions in the distribution. Therefore, the above operation makes it possible to execute classification with an appropriate permissible value X for each peak.

Referring back to FIG. 4, based on the process result by the aligner 306, the average calculator 309 calculates the average of mass-to-charge ratios and the average of detected intensities of data items included in the peak clusters (Step S6). Here, the average of mass-to-charge ratios is calculated by dividing the total value of the mass-to-charge ratios of the data items included in the clusters, by the number of data items (the number of observation times). The average of detected intensities is calculated by dividing the total value of the detected intensities of the data items included in the clusters by the number of MS spectrums N (the number of measurement times). This is based on a physical interpretation that if not observed, the detected intensity is zero.

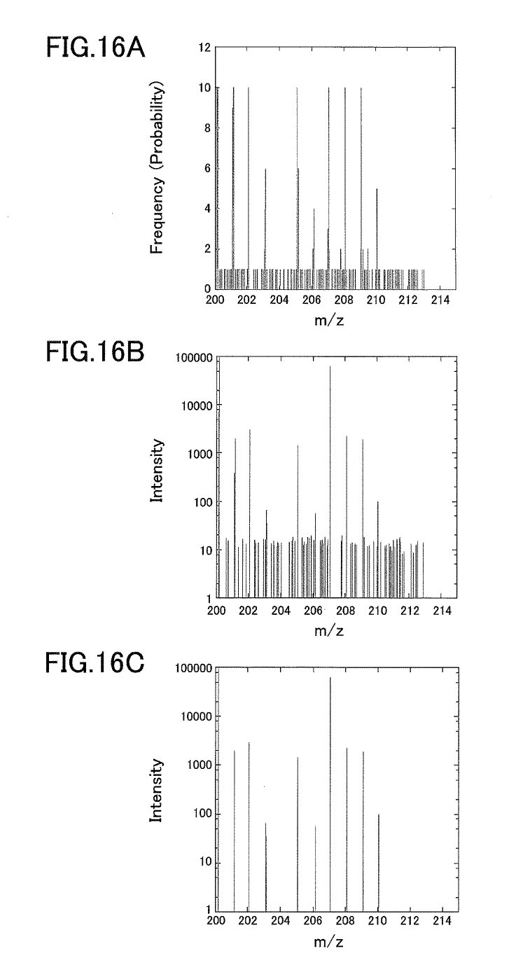

Next, the statistical information calculation and noise removal unit 310 calculates, as one of the statistical information items, a detection frequency from the ratio of the number of the data items included in each cluster to the number of MS spectrums, and based on the detection frequency, removes a peak cluster including unreliable data items as noise or the like (Step S7). FIGS. 16A-16C are diagrams illustrating an example of noise removal. FIG. 16A illustrates the detection frequency with respect to the mass-to-charge ratio before noise removal, FIG. 16B illustrates the detected intensity with respect to the mass-to-charge ratio before noise removal, and FIG. 16C illustrates the detected intensity with respect to the mass-to-charge ratio after noise removal. In FIG. 16A, peaks having small detection frequencies densely appearing close to the lower side can be determined as noise. By removing the corresponding peaks in FIG. 16B, a simple average MS spectrum without the noise can be obtained as illustrated in FIG. 16C. Note that what is important here is that noise removal is executed based on the detection frequency, not dependent on the detected intensity. Therefore, even if a peak has a small detected intensity, the peak is not determined as noise as long as the peak has a high detection frequency, and remains as a meaningful measurement result.

Moreover, the detection frequency (observation probability) reflects the probability of existence of a substance to be examined, and can be used for evaluating the deviation of the substance in the sample 2.

Referring back to FIG. 4, the data output unit 311 outputs a data group that includes data items of pairs of the average of the mass-to-charge ratios and the average of the detected intensities of each peak cluster after noise has been removed, as data of the average MS spectrum (Step S8). Note that the data output unit 311 may output the average MS spectrum processed to have a graph format.

<Applications>

The embodiment described above has no limitation about objects to which mass spectrometry is applied, and the mass spectrometry can be applied to, for example, a human cell molecule (a substance extracted from the inside of a human cell), to use the result for supporting diagnosis by a doctor.

Moreover, although the embodiment described above has been described for cases where the MS spectrums is processed, it is applicable not only to processing the MS spectrums but also to processing discrete spectrums, for example, optical spectrums (infrared spectrums, ultraviolet spectrums, etc.) and nuclear magnetic resonance spectrums.

<Summary>

As has been described so far, according to the embodiments, it is possible to raise the precision of data classification.

Thus, the present invention has been described with the preferable embodiments. Although the specific examples have been illustrated and described here, it is obvious that the specific examples can be modified and change din various ways without deviating from the scope of the present invention defined in the claims. In other words, the present invention should not be taken to be limited by details of the specific examples and the attached drawing.

Note that the “mass-to-charge ratio (m/z)” is an example of a “physical index value”. The “MS spectrum” is an example of a “data group”. The “spectrum number” is an example of “identification information”.

Further to the description so far, the following additional remarks are disclosed.

Additional remark 1. A method for classifying data, executed by a computer, the method comprising:
obtaining a plurality of data groups, each of the data groups including a plurality of data items about detected intensities being associated with physical index values, respectively; and
classifying, base don identification information of each of the data groups and the physical index values, the data items included in the data groups into a plurality of clusters.

Additional remark 2. The method for classifying data as described in Additional remark 1, wherein the classifying classifies the data items included in the data groups into the clusters so that no duplication of the identification information is generated among the data items included in each of the clusters.

Additional remark 3. The method for classifying data as described in Additional remark 1 or 2, the method further comprising:
executing a calculation about the detected intensities for each of the clusters.

Additional remark 4. The method for classifying data as described in Additional remark 3, wherein the calculation is to calculate an average of the detected intensities for each of the clusters.

Additional remark 5. The method for classifying data as described in any Additional remarks 1 to 4, the method further comprising:
outputting, based on a number of the data groups and a number of the data items included in each of the clusters, information about evaluation of the data items included in each of the clusters.

Additional remark 6. The method for classifying data as described in any one of Additional remarks 1 to 5, wherein the data groups are obtained as a result of mass spectrometry applied one or more times to an object sample,
wherein the data items about the detected intensities are about amounts of detected substances included in the sample,
wherein the physical index value is an index value about mass of the substance.

Additional remark 7. The method for classifying data as described in Additional remark 6, wherein the sample is constituted with substances existing in a human cell.

Additional remark 8. A method for classifying data, executed by a computer, the method comprising:
obtaining a plurality of measurement data items obtained by applying a same analytical technique for a plurality of times; and
clustering measurement values of physical index values obtained by the plurality of items of the measurement, depending on similarity of the physical index values, and based on measurement identification information identifying which of the measurement with which the measurement value has been obtained, so as not to have the measurement value obtained by the same measurement to be included in a same group.

Additional remark 9. A method for classifying data, executed by a computer, the method comprising:
obtaining a plurality of data groups, each of the data groups including a plurality of data items about detected intensities being associated with index values about mass of a plurality of substances, respectively; and
classifying, base don identification information of each of the data groups and the index values about the mass of the substances, the data items included in the data groups into a plurality of clusters.

Additional remark 10. A data classification apparatus, comprising:
a unit configured to obtain a plurality of data groups, each of the data groups including a plurality of data items about detected intensities being associated with physical index values, respectively; and
a unit configured to classify, based on identification information of each of the data groups and the physical index values, the data items included in the data groups into a plurality of clusters.

Additional remark 11. The data classification apparatus as described in Additional remark 10, wherein the classifying classifies the data items include din the data groups into the clusters so that no duplication of the identification information is generated among the data items included in each of the clusters.

Additional remark 12. The data classification apparatus as described in Additional remark 10 or 11, the method further comprising:
executing a calculation about the detected intensities for each of the clusters.

Additional remark 13. The data classification apparatus as described in Additional remark 12, wherein the calculation is to calculate an average of the detected intensities for each of the clusters.

Additional remark 14. The data classification apparatus as described in any Additional remarks 10 to 13, the method further comprising:
outputting, based on a number of the data groups and a number of the data items included in each of the clusters, information about evaluation of the data items included in each of the clusters.

Additional remark 15. The data classification apparatus as described in any one of Additional remarks 10 to 14, wherein the data groups are obtained as a result of mass spectrometry applied one or more times to an object sample,
wherein the data items about the detected intensities are about amounts of detected substances included in the sample,
wherein the physical index value is an index value about mass of the substance.

Additional remark 16. The data classification apparatus as described in Additional remark 15, wherein the sample is constituted with substances existing in a human cell.

Additional remark 17. A data classification apparatus, executed by a computer, the method comprising:
obtaining a plurality of measurement data items obtained by applying a same analytical techniques for a plurality of times; and
clustering measurement values of physical index values obtained by the plurality of times of the measurement, depending on similarity of the physical index values, and based on measurement identification information identifying which of the measurement with which the measurement value has been obtained, so as not to have the measurement value obtained by the same measurement to be included in a same group.

Additional remark 18. A data classification apparatus, executed by a computer, the method comprising:
obtaining a plurality of data groups, each of the data groups including a plurality of data items about detected intensities being associated with index values about mass of plurality of substances, respectively; and
classifying, based on identification information of each of the data groups and the index values about the mass of the substances, the data items included in the data groups into a plurality of clusters.

Additional remark 19. A non-transitory computer-readable recording medium having a program stored therein for causing a computer to execute a process for classifying data, the process comprising:
obtaining a plurality of data groups, each of the data groups including a plurality of data items about detected intensities being associated with physical index values, respectively; and
classifying, based on identification information of each of the data groups and the physical index values, the data items included in the data groups into a plurality of clusters.

Additional remark 20. The non-transitory computer-readable medium as described in Additional remark 19, wherein the classifying classified the data items included in the data groups into the clusters so that no duplication of the identification information is generated among the data items included in each of the clusters.

Additional remark 21. The non-transitory computer-readable medium as described in Additional remark 19 or 20, the method further comprising:
executing a calculation about the detected intensities for each of the clusters.

Additional remark 22. The non-transitory computer-readable medium as described is Additional remark 21, whrein the calculation is to calculate an average of the detected intensities for each of the clusters.

Additional remark 23. The non-transitory computer-readable medium as described in any Additional remarks 19 to 22, the method further comprising:
outputting, based on a number of the data groups and a number of the data items included in each of the clusters, information about evaluation of the data items included in each of the clusters.

Additional remark 24. The non-transitory computer-readable medium as described in any one of Additional remarks 19 to 23, wherein the data groups are obtained as a result of mass spectrometry applied one or more times to an object sample,
wherein the data items about the detected intensities are about amounts of detected substances included in the sample,
wherein the physical index value is an index value about mass of the substance.

Additional remark 25. The non-transitory computer-readable medium as described in Additional remark 24, wherein the sample is constituted with substances existing in a human cell.

Additional remark 26. A non-transitory computer-readable medium having a program stored therein for causing a computer to execute a process for classifying data, the process comprising:
obtaining a plurality of measurement data items obtained by applying a same analytical technique for a plurality of times; and
clustering measurement values of physical index values obtained by the plurality of times of the measurement, depending on similarity of the physical index values, and based on measurement identification information identifying which of the measurement with which the measurement value has been obtained, o as not to have the measurement value obtained by the same measurement to be included in a same group.

Additional remark 27. A non-transitory computer-readable medium having a program stored therein for causing a computer to execute a process for classifying data, the process comprising:
obtaining a plurality of data groups, each of the data groups including a plurality of data items about detected intensities being associated with index values about mass of a plurality of substances, respectively; and
classifying, based on identification information of each of the data groups and the index values about the mass of the substances, the data items included in the data groups into a plurality of clusters.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A method for classifying data, executed by a computer, the method comprising:
obtaining a plurality of data groups, each of the data groups including a plurality of data items about detected intensities being associated with physical index values, respectively; and
classifying, based on identification information of each of the data groups and the physical index values, the data items included in the data groups into a plurality of clusters.

2. The method of classifying data as claimed in claim 1, wherein the classifying classifies the data items included in the data groups into the clusters so that no duplication of the identification information is generated among the data items included in each of the clusters.

3. The method for classifying data as claimed in claim 1, the method further comprising:
executing a calculation about the detected intensities for each of the clusters.

4. The method for classifying data as claimed in claim 3, wherein the calculation is to calculate an average of the detected intensities for each of the clusters.

5. The method for classifying data as claimed in claim 1, the method further comprising:
outputting, based on a number of the data groups and a number of the data items included in each of the clusters, information about evaluation of the data items included in each of the clusters.

6. The method of classifying data as claimed in claim 1, wherein the data groups are obtained as a result of mass spectrometry applied one or more times to an object sample,
wherein the data items about the detected intensities are about amounts of detected substances included in the sample,
wherein the physical index value is an index value about mass of the substance.

7. The method for classifying data as claimed in claim 6, wherein the sample is constituted with substances existing in a human cell.

8. A data classification apparatus, comprising:
a processor that executes a process including:
obtaining a plurality of data groups, each of the data groups including a plurality of data items about detected intensities being associated with physical index values, respectively; and
classifying, based on identification information of each of the data groups and the physical index values, the data items included in the data groups into a plurality of clusters.

9. A non-transitory computer-readable recording medium having a program stored therein for causing a computer to execute a process for classifying data, the process comprising:
obtaining a plurality of data groups, each of the data groups including a plurality of data items about detected intensities being associated with physical index values, respectively; and
classifying, based on identification information of each of the data groups and the physical index values, the data items included in the data groups into a plurality of clusters.
